(19) United States
(12) Patent Application Publication
(10) Pub. No.: US 2022/0406471 A1
(43) Pub. Date: Dec. 22, 2022

(54) PATHOGENIC VECTOR DYNAMICS BASED ON DIGITAL TWIN

(71) Applicant: INTERNATIONAL BUSINESS MACHINES CORPORATION, Armonk, NY (US)

(72) Inventors: Partho Ghosh, Gangnapur (IN); Saraswathi Sailaja Perumalla, Visakhapatnam (IN); Sri Satya Trinadha Narasimha Rao Pyla, Visakhapatnam (IN); Pavan K Manda, Visakhapatnam (IN)

(21) Appl. No.: 17/353,286

(22) Filed: Jun. 21, 2021

(51) Int. Cl. G16H 50/80; G16H 40/67; G16H 50/70; G16H 50/50; G16H 50/20; G16H 50/30; G06N 5/04; G06N 20/00

(52) U.S. Cl. CPC G16H 50/80; G16H 40/67; G16H 50/70; G16H 50/50; G16H 50/20; G16H 50/30; G06N 5/04; G06N 20/00; H04L 67/12

(57) ABSTRACT

Epidemiological modeling and simulation includes generating a virtual representation of a predetermined geospatial region by communicating in real time via a data communications network with a plurality of sensor-endowed computing nodes that capture and convey sensor-generated data in real time to the networked computer. A causal network is derived for mapping biodata onto inferences regarding pathogenic vector dynamics of a known pathogen based on stochastic vector probabilities. The biodata can be culled from historical data associated with the predetermined geospatial region. A visualization of selected characteristics of the predetermined geospatial region is generated and a corresponding epidemiological model created based on the historical data. Sensor-generated data and historical data are correlated. An expected effect of the known pathogen on a population of the geospatial region is predicted using a model simulation based on correlating the sensor-generated data and historical data.

### BACKGROUND

This disclosure relates to computer modeling and simulation, and more particularly to modeling and simulating epidemiological scenarios and outcomes.

Population size and density has increased in many regions of the world, in many places dramatically. The increased size and density of populations coupled with widespread means of travel of large numbers of people between regions has heightened concerns over the spread of infectious diseases. Increasingly, public health officials and others rely on computer-implemented mathematical models to project how infectious diseases progress and to devise public health strategies for controlling the spread of infectious diseases and mitigating their consequences.

### SUMMARY

In one or more embodiments, a method includes generating a virtual representation of a predetermined geospatial region by communicating in real time via a data communications network with a plurality of sensor-endowed computing nodes that capture and convey sensor-generated data in real time to a networked computer. The method includes deriving a causal network for mapping biodata onto inferences regarding pathogenic vector dynamics of a known pathogen based on stochastic vector probabilities, wherein the biodata is culled from historical data associated with the predetermined geospatial region. The method includes generating a visualization of selected characteristics of the predetermined geospatial region and creating a corresponding epidemiological model based on the historical data. The method includes correlating the sensor-generated data and historical data. The method includes predicting an expected effect of the known pathogen on a population of the geospatial region using a model simulation based on correlating the sensor-generated data and historical data.

In one or more embodiments, a system includes a processor configured to initiate operations. The operations include generating a virtual representation of a predetermined geospatial region by communicating in real time via a data communications network with a plurality of sensor-endowed computing nodes that capture and convey sensor-generated data in real time to a networked computer. The operations include deriving a causal network for mapping biodata onto inferences regarding pathogenic vector dynamics of a known pathogen based on stochastic vector probabilities, wherein the biodata is culled from historical data associated with the predetermined geospatial region. The operations include generating a visualization of selected characteristics of the predetermined geospatial region and creating a corresponding epidemiological model based on the historical data. The operations include correlating the sensor-generated data and historical data. The operations include predicting an expected effect of the known pathogen on a population of the geospatial region using a model simulation based on the correlating the sensor-generated data and historical data.

In one or more embodiments, a computer program product includes one or more computer readable storage media having instructions stored thereon. The instructions are executable by a processor to initiate operations. The operations include generating a virtual representation of a predetermined geospatial region by communicating in real time via a data communications network with a plurality of sensor-endowed computing nodes that capture and convey sensor-generated data in real time to a networked computer. The operations include deriving a causal network for mapping biodata onto inferences regarding pathogenic vector dynamics of a known pathogen based on stochastic vector probabilities, wherein the biodata is culled from historical data associated with the predetermined geospatial region. The operations include generating a visualization of selected characteristics of the predetermined geospatial region and creating a corresponding epidemiological model based on the historical data. The operations include correlating the sensor-generated data and historical data. The operations include predicting an expected effect of the known pathogen on a population of the geospatial region using a model simulation based on the correlating the sensor-generated data and historical data.

This Summary section is provided merely to introduce certain concepts and not to identify any key or essential features of the claimed subject matter. Other features of the inventive arrangements will be apparent from the accompanying drawings and from the following detailed description.

### BRIEF DESCRIPTION OF THE DRAWINGS

The inventive arrangements are illustrated by way of example in the accompanying drawings. The drawings, however, should not be construed to be limiting of the inventive arrangements to only the particular implementations shown. Various aspects and advantages will become apparent upon review of the following detailed description and upon reference to the drawings.

FIG. 1 illustrates an example epidemiological modeling and simulation system.

FIG. 2 illustrates certain methodological aspects implemented by the system of FIG. 1.

FIG. 3 illustrates methodological aspects implemented by the system of FIG. 1 using a digital twin.

FIG. 4 illustrates methodological aspects implemented by the system of FIG. 1 using a digital twin comprising multiple interactive sub-twins.

FIG. 5 depicts a cloud computing environment.

FIG. 6 depicts abstraction model layers of the cloud computing environment of FIG. 5.

FIG. 7 depicts a cloud computing node which can implement the system of FIG. 1.

### DETAILED DESCRIPTION

While the disclosure concludes with claims defining novel features, it is believed that the various features described within this disclosure will be better understood from a consideration of the description in conjunction with the drawings. The process(es), machine(s), manufacture(s) and any variations thereof described herein are provided for purposes of illustration. Specific structural and functional details described within this disclosure are not to be interpreted as limiting, but merely as a basis for the claims and as a representative basis for teaching one skilled in the art to variously employ the features described in virtually any appropriately detailed structure. Further, the terms and phrases used within this disclosure are not intended to be limiting, but rather to provide an understandable description of the features described.

This disclosure relates to computer modeling and simulation, and more particularly to modeling and simulating epidemiological scenarios and outcomes. Forecasting the progression and effects of pathogen-fueled pandemic typically requires an understanding of the pathogen vector dynamics, that is, the behavior, influence, and progression of a pathogen within a given geospatial region (e.g., city, town, country). Such an understanding aids in devising mitigation strategies to slow the progression and contain the spread of the pathogen. Public health experts increasingly rely on evidence-based modeling using information from multiple sources. An epidemiological model's accuracy often depends on acquiring extensive data from as many diverse sources of information as possible. However, acquiring and using such data, in practice, can be difficult. Thus, there is an inevitable potential that critical data is missed. Delayed or inaccurate data regarding pathogenic vector dynamics, however, can hamper needed epidemiological modeling and simulation, thus impeding health preparedness and disease mitigation strategies. The result can be an extensive transmission of a known pathogen—often leading to an epidemic.

The methods, systems, and computer program product disclosed herein rapidly and efficiently acquire real-time and near real-time data from a wide variety of dispersed sources via wired and/or wireless network connections. Using the data, the methods, systems, and computer program product disclosed can generate digital models of corresponding real-world epidemiological events and simulate—also in real- or near real-time—epidemiological outcomes. One aspect of the arrangements disclosed herein, is the virtual representation of a geospatial region generated using data acquired via a data communications network in real time from multiple sensor-endowed computing nodes.

The virtual representation, in various arrangements, is a virtual twin of the geospatial region. As defined herein, a “digital twin” is a logical construct, implemented with a computer, that connects a physical (real) object and a digital (virtual) object, the connections comprise data generated by the physical object conveyed to the digital object, and can include information produced by the digital object that is conveyed to the physical object. In the present context, the connective data and information can be used for modeling and simulating epidemiological outcomes.

In certain arrangements disclosed herein, a digital twin comprises multiple interacting sub-twins. The use of digital twins eliminates fundamental constraints related to obtaining data related to time and place based on human observation and allows for the decoupling of physical flows of information concerning population movements, density, sanitization operations, healthcare protocols, and the like allowing for more rapid epidemiological assessments. Furthermore, digital twins can not only represent current physical states, but can also reproduce historical states and simulate future states. One aspect of digital representation of states is the ability to train artificial intelligence (AI) models using machine learning in which the digital representations provide training and validation data, obviating the time and expense of gathering data from dispersed physical sources.

Another aspect of the methods, systems, and computer program product disclosed herein, is derivation of a causal network for mapping biodata represented digitally by the digital twin of a geospatial region (e.g., community, town, city, country) onto inferences regarding pathogenic vector dynamics of a known pathogen and threshold values based on stochastic vector probabilities. As defined herein, a “pathogenic vector” is an organism that can transmit infectious pathogens (substances capable of causing disease) between humans or from animal to humans. As “Pathogenic vector dynamics,” as defined herein, refer to time-based changes in one or more pathogenic vectors and outcomes related thereto that occur over a predefined time interval. A “causal mapping,” as defined herein, is a network of nodes corresponding to factors which are connected or linked by edges or arcs. The edges or arcs (directed or non-directed) represent causality (e.g., a causal relationship) between one or more factors and one or more other factors in the sense that such that a link between one factor and another factor indicates that one is caused by the other. In the present context, causality can be based, for example, on statistical inference or epidemiological methodology and can be represented by a causal mapping corresponding to a probability that a factor (e.g., pathogenic vector, weather pattern, population pattern, social condition) causes one or more outcomes (e.g., spread of a pathogen within a designated region).

A stochastic vector (also referred to as a multivariate random variable), is a vector whose elements are each a random variable. The degree of statistical certainty regarding a causal inference can be expressed probabilistically by a stochastic vector. Derivations of causal networks using various epidemiological and statistical techniques can be performed by the methods, systems, and computer program product disclosed during distinct phases. Distinct phases can include, for example, phases during which an epidemic or outbreak of a disease endemic to particular population or region is intensifying, or during eradication phases when such an outbreak is receding.

Further aspects of the embodiments described within this disclosure are described in greater detail with reference to the figures below. For purposes of simplicity and clarity of illustration, elements shown in the figures have not necessarily been drawn to scale. For example, the dimensions of some of the elements may be exaggerated relative to other elements for clarity. Further, where considered appropriate, reference numbers are repeated among the figures to indicate corresponding, analogous, or like features.

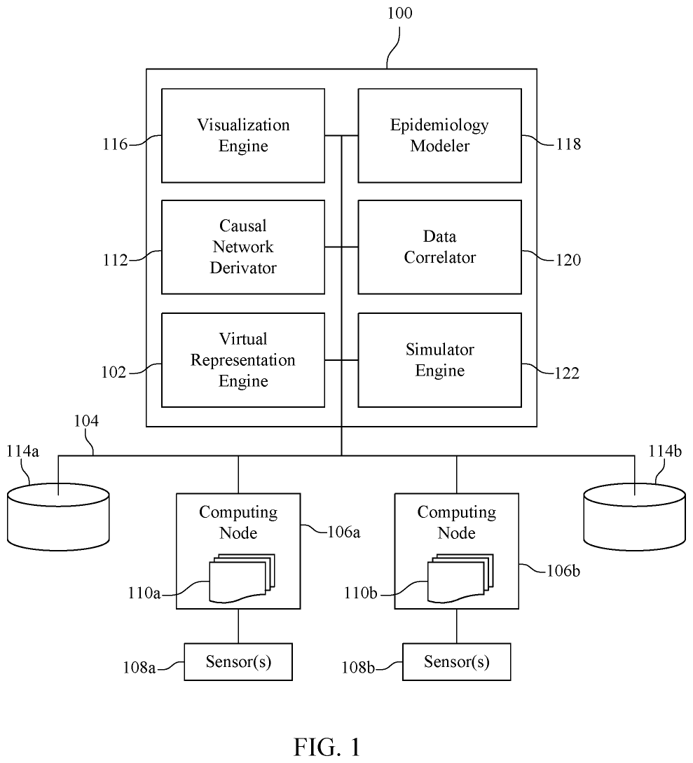

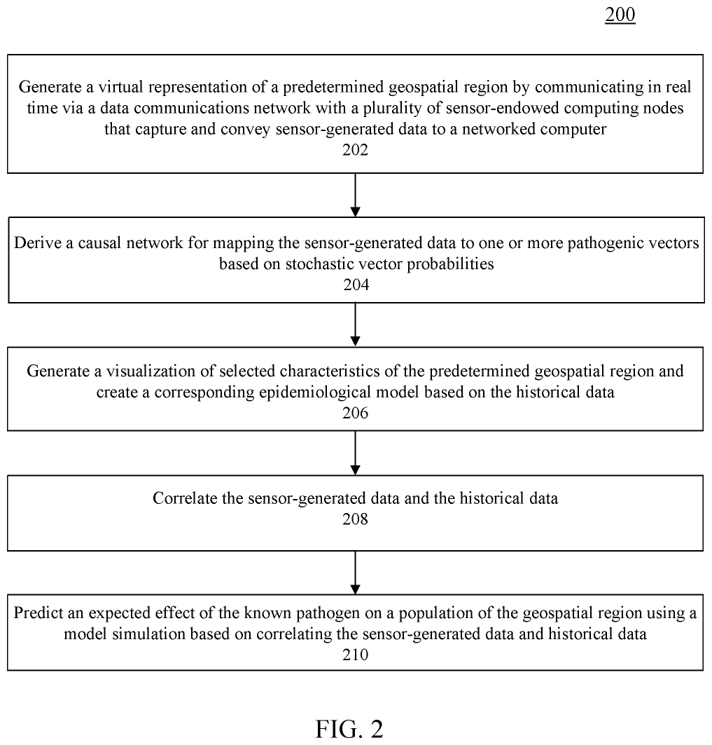

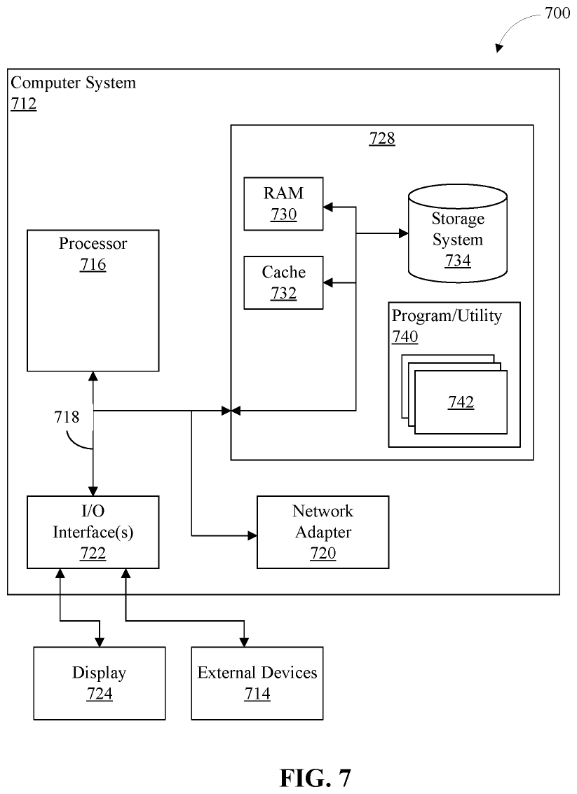

FIGS. 1 and 2, respectively, illustrate example epidemiological simulation and modeling (ESM) system 100 and methodology 200, which implements certain aspects of ESM system 100. ESM system 100, illustratively, can be implemented in computer-executable instructions and/or dedicated circuitry. In various arrangements, ESM system 100 is implemented in a computing node (e.g., cloud-based server) such as computing node 700 (FIG. 7).

At block 202, virtual representation engine 102 generates a virtual representation of a predetermined geospatial region. The geospatial region can be, for example, a community, a city, a state, or a country. Virtual representation engine 102 generates the virtual representation of a geospatial region by communicating, in real time via data communications network 104, with multiple computing nodes 106a and 106b, each of the computing nodes being endowed respectively with one or more sensors 108a and 108b. Although illustratively only two computing nodes are shown, each operatively coupled with a single sensor, it is to be understood that ESM system 100 can communicatively couple to a larger number of computing nodes, each operatively coupled with a variable number of sensors. ESM system 100 receives sensor-generated data 110a and 110b from sensor-endowed computing nodes 106a and 106b, sensor-generated data 110a and 110b captured by sensors 108a and 108b, respectively, in real time. Virtual representation engine 102 generates the virtual representation using sensor-generated data 110a and 110b.

At block 204, causal network derivator 112 derives a causal network for mapping biodata onto inferences regarding pathogenic vector dynamics of a known pathogen based on stochastic vector probabilities. The biodata is culled from historical data associated with the predetermined geospatial region and electronically stored in databases 114a and 114b, which ESM system 100 accesses electronically via data communications network 104. While only two such databases are shown, it is to be understood that ESM system 100 can access a greater or lesser number of databases via a wired or wireless connection for acquiring various historical data. The stochastic vector probabilities can be calculated by causal network derivator 112 during times that an epidemic outbreak or outbreak of a disease or condition endemic to particular population or regions is intensifying. The stochastic vector probabilities also can be derived during eradication phases when such an outbreak is receding.

At block 206, visualization engine 116 generates a visualization (e.g., 3-D visual imaging) of selected characteristics of the predetermined geospatial region based on various historical data electronically stored in databases 114a and 114b and accessible by ESM system 100 via data communications network 104. Based on the historical data, epidemiology modeler 118 creates a corresponding epidemiological model based on the historical data.

At block 208, data correlator 120 correlates the sensor-generated data and the historical data. At block 210, based on correlated sensor-generated data and historical data, simulator engine 122 predicts an expected effect of the known pathogen on a population of the geospatial region using a simulation model.

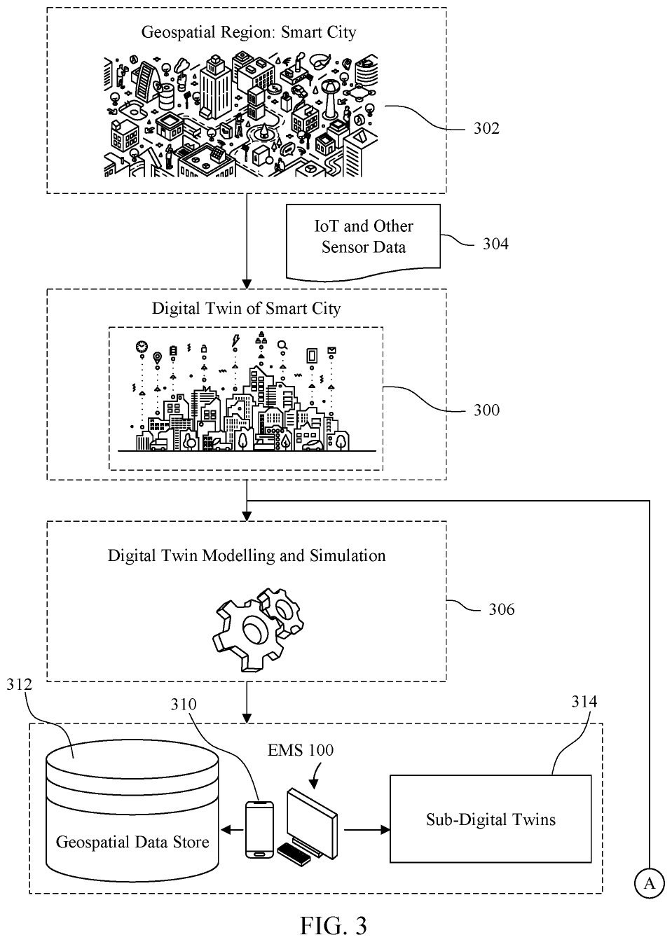

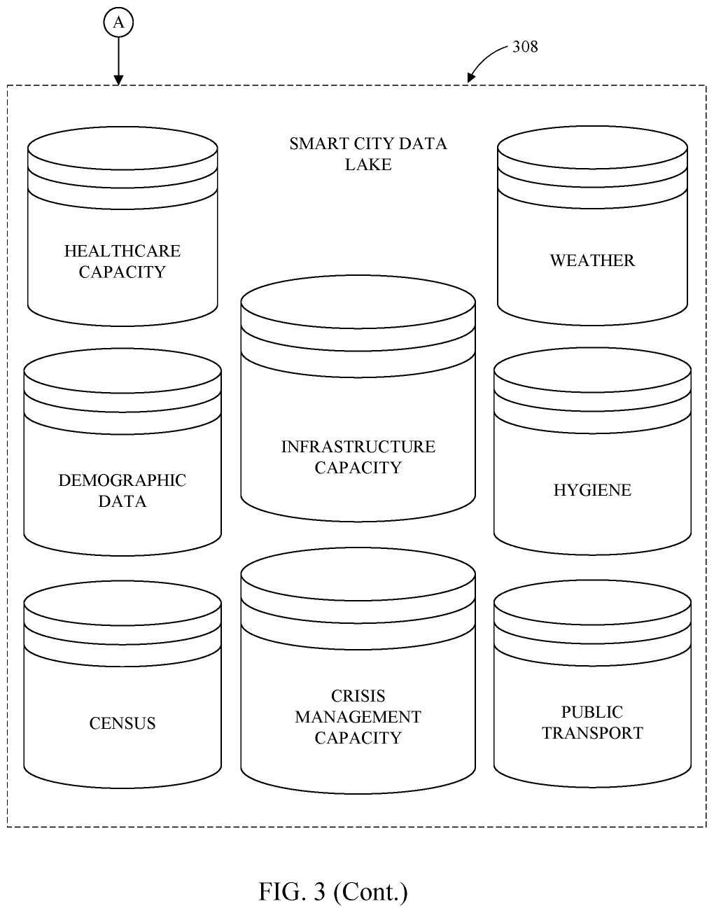

In certain arrangements, the virtual representation generated by virtual representation engine 102 is a digital twin of the geospatial region. Referring additionally to FIG. 3, virtual representation engine 102 generates a virtual representation comprising digital twin 300. Digital twin 300 is twined with smart city 302, from which IoT and other real-time sensor data 304 is retrieved via data communications network 104 for performing modeling and simulation 306 of the digital twin. IoT and other real-time sensor data 304 is electronically stored in a smart city data lake comprising a plurality of computer databases 308. Computer system 310 implements ESM 100, storing geospatial data in geospatial data store 312. In certain arrangements, as illustrated, the digital twin generated by virtual representation engine 102 is a digital twin comprising plurality of interacting sub-twins 314.

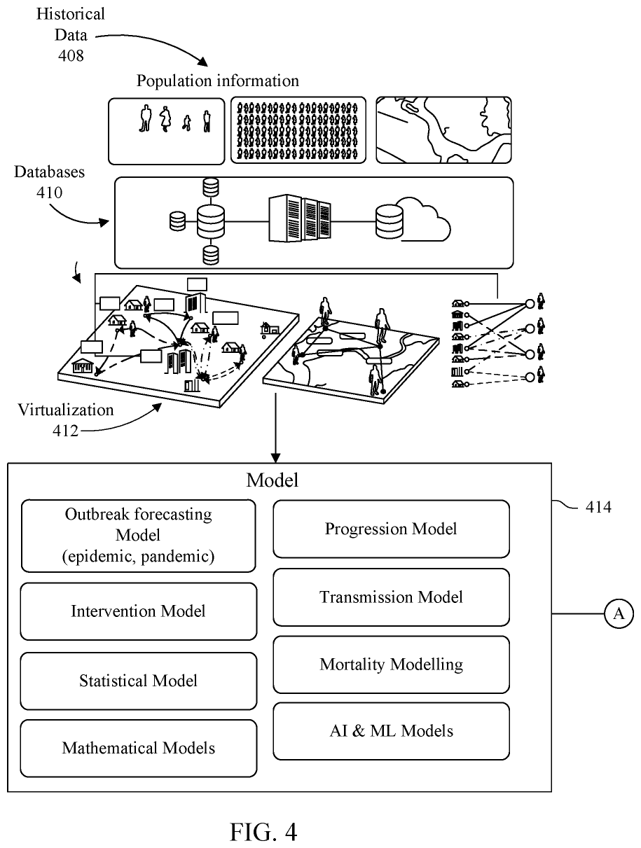

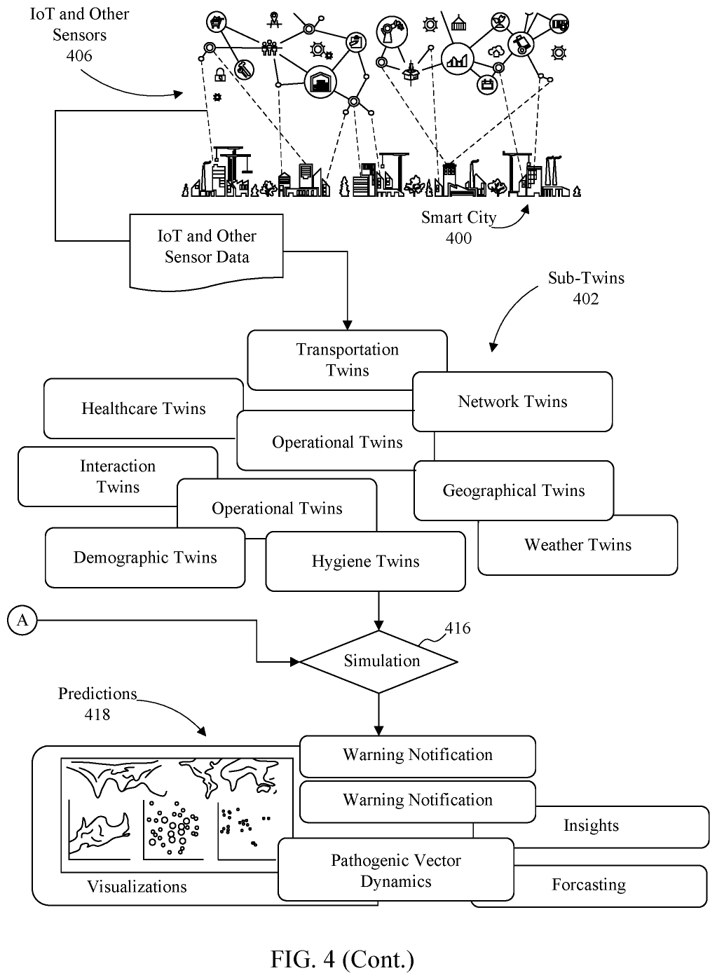

Referring still to FIGS. 1 and 2 and additionally now to FIG. 4, a more extensive view is provided in which the virtual representation of a geospatial region (smart city 400) is a digital twin comprising plurality of interacting digital sub-twins 402. Digital sub-twins 402 are generated by virtual representation engine 102 based on IoT and other sensor data 404 acquired by IoT and other sensors 406 of smart city 400. Various types of historical data 408 can be acquired from one or more databases 410. In addition to biodata corresponding to smart city 400, historical data 408 can include, for example, weather data, healthcare data, census data, demographic information, and a host of other data specific to smart city 400. Other historical data 408 can include, for example, medical testing data (e.g., serological results, vaccination histories) on members of the population, geographic data, climate conditions, air quality, levels of pollution, various population and other studies, and emergency and/or crisis management data.

Causal network derivator 112 derives a causal network to map biodata onto inferences regarding pathogenic vector biodata onto inferences regarding pathogenic vector dynamics of a known pathogen based on stochastic vector probabilities. Visualization engine 116 generates one or more visualizations 412, which illustratively are 3-D visual images of selected characteristics of smart city 400. Epidemiology modeler 118 creates one or more epidemiological models 414 based on historical data 408, the one or more models can mathematical models and statistical models, as well as AI and machine learning models.

Data correlator 120 correlates IoT and other sensor data 404 with historical data 408. Based on one or more models 416 created by epidemiology modeler 118, simulator engine 122 performs simulation 416, generating one or more predictions 418 based on inputting IoT and other sensor data 404 correlated with historical data 408.

Data correlators 120 can correlate various kinds of data. The data correlated include, for example, data pertaining to infrastructure units (e.g., sewage system), pollution levels, population demographics (e.g., density and distribution within the geospatial region), social patterns at various times of day (e.g., work, school, shopping), population mobility patterns via public and private transport, age distribution, and population inflow/outflow via an infrastructure unit (e.g., airport, railway, city metro). Data correlated can include data on food distribution infrastructures like hotels, street-side food stalls, mobile food vans, vegetable markets, wet markets, packaged food markets, and the like. Data can be collated with healthcare demographics (e.g., vaccination histories), ratios of pre-existing health condition, amounts of health spending by by age, gender or other characteristics. Healthcare data can include hospital capacity for the geospatial region.

In various arrangements, a digital twin created by virtual representation engine 102 exchanges data (e.g., sensor-generated data) and information between virtual (non-physical) entities and physical entities with the geospatial region (e.g., smart city), as described above, relevant to determining values and/or thresholds of various pathogenic vectors. The pathogenic vectors correspond, for example, to pathogenic infection symptoms, case morbidity rates, degree of spread ability, serial interval rate, close-contact proximity, transmission rates, symptom onset periods, transmission modes, aggravating pre-existing medical conditions, and most vulnerable patients according to gender, age, and/or pre-existing medical condition.

A digital twin created by virtual representation engine 102, in some arrangements, can be a composite of multiple, discrete digital twins corresponding to an entity comprising multiple components. The composite can be an assembly or system twin that comprises multiple assembly twins.

In some arrangements, a digital twin created by virtual representation engine 102 can comprise a network for acquiring data, with which epidemiology modeler 118 creates, for example, a secondary contact tracing model based on location data and pathogenic vector dynamics and their corresponding values. Epidemiology modeler 118 likewise can create a stochastic transmission model, parameterized to a pathogenic outbreak (e.g., Covid19, Ebola, Spanish flu, avian flu) for quantifying potential covariates, such as a threshold for classifying close contact, basic reproduction number, Ro, susceptible reproduction factor, RoS (S being the susceptible fraction of population), incubation period from symptom onset to isolation of an individual, proportion of transmissions occurring before symptom onset, and/or proportion of sub-clinical infections.

Simulator engine 122 can simulate various different epidemiological aspects. The simulations can be used to predict epidemiological outcomes, from which conclusions can be drawn and corresponding threshold values determined. The outcomes, conclusions, and threshold values relate to pathogenic vectors like pathogenic infection symptoms, case morbidity rate, likely degree of spread, serial interval rate, close-contact proximity, reproduction factors, susceptibility factors, epidemic peak within the geospatial region, and/or combination of regional transmission rates for the entire geospatial region. Additionally, simulator engine 122 can further simulate pathogenic-population vectors. The pathogenic-population vectors include, for example, number of likely encounters between individuals, average travel radius of members of the population, expected recovery periods and recovery rates, incubation periods, symptom onset periods, days with symptoms exhibited by infected individuals, pathogen transmission modes, potentially aggravating pre-existing medical conditions, individuals deemed most vulnerable according to gender, age and/or pre-existing medical condition, predictions regarding herd immunity, and identification of patient zero within the geospatial region.

The simulations generated by simulator engine 122 can be used to determine dynamic contact rates, identify disease transmission patterns, transmission trajectories, surge periods, outbreak progression and related characteristics by simulating spatial contact models for a heterogeneous population. In addition to demographic factors, the simulation can incorporate weather-related factors, population vaccine histories, and density of the geospatial region. By correlating the factors with the pathogen vector dynamics, a scaling of directly transmitted pathogens in a sparse spectrum of densities and frequencies of population mobility patterns can be generated to describe various forms of pathogen transmissions within the geospatial region. The simulations can be used to formulate time-bound pathogenic transmission trajectories for the geospatial region, describe disease progression and decay of the reproduction factor and thereby determine characteristics during a peak pandemic period, such as peaking duration, infection fatality rates, daily fatality rates, daily infection rates, and probabilities of infection, for example.

Simulator engine 122 can simulate daily weather patterns, based on time of day, thereby automatically generating insights into how weather changes contact rates and transmission patterns with respect to the geospatial region. For example, changing contact patterns collated with warmer weather may be shown to slow the spread of the pathogen, thereby reducing the geospatial pathogenic Ro. Monsoon seasons or heavy rain patterns may be shown to increase the pathogenic Ro. Disease transmission may be shown to be high during evening and nighttime hours for a sub-region, for example. Simulator engine 122 can simulate the geospatial pathogenic vectors with dynamic contextual economic and population scenarios to derive mitigation efficacy insights pertaining to various non-pharmaceutical interventions (NPI) strategies.

The model simulation of simulator engine 112 simulates outcomes of a predefined NPI strategy for determining an effectiveness threshold of the non-pharmaceutical intervention strategy. NPI strategies can focus on disease control mechanisms such as cyclic mitigation, periods of no intervention, cyclic suppression, cyclic relaxation, relaxed mitigation, social distancing, percentile quarantines, social distancing, time-bound mitigations, population-based mitigation, mandatory isolation, transport-based mitigation, and event-based mitigation, for example.

The likely effectiveness of healthcare-worker interventions can be determined by simulator engine 112 simulating how pathogen transmission and reproductive rates, the number of cases, and the like change with one or more NPI strategies. The simulation also can identify various adverse mental and sedentary behavioral effects on the geospatial region's population using dynamic statistical survey sampling during a disease outbreak or pandemic. Using specific historical data pertaining to geospatial region's healthcare system and related factors such as equipment manufacturing, logistics and other historical data, simulation engine 112 can generate a geographically discrete simulation healthcare model that correlates pathogenic vectors with current capacity of the healthcare system and dynamically forecasts various factors regarding the healthcare system such as hospital capacity, needed testing kits, leeway time for testing the population, and amount of population to be tested. The simulation engine 112, based on one or more related simulations, can forecast healthcare system factors. The forecast can be based on the sensor-generated data correlated with the historical data. The simulations can provide insights for pool testing of a designated “hotspot.” The simulations can further determine routine variations in capacity strain on healthcare processes, clinical outcomes, resource constraints and identify critical medical devices (e.g., ventilators, oxygen supplies, test kits, PPE kits).

In certain arrangements, epidemiology modeler 118 generates one or more AI models using machine learning (e.g., deep neural network, support vector machine, Bayesian network). One advantage of ESM system 100 is the AI model(s) can be trained and validated using simulated input generated by simulator engine 112. Using simulated data for training and validation of the AI model(s) obviates the time and resources needed to obtain training and validation data by physical observation or extensive data collection from disparate, often remotely located, databases. One trained and validated, such an AI model can be used for various purposes. For example, in one arrangement, an AI model predicts risk factors associated with pre-existing conditions of members of a population.

In another arrangement, epidemiology modeler 118, using for example a model trained with machine learning, can generate geospatial time series-based data that visualization engine 116 uses to generate a visualization of pathogenic and/or pandemic characteristics (e.g., pandemic/epidemic infection curve, death rates, mitigation efficacy). The visualization can further be used to derive insights for effective mitigation, health warning and alerts, and control mechanisms. Relatedly, simulator engine 112 can simulate the portion of the geospatial region's population to be tested. The simulation can use, for example, IoT feeds from sensors monitoring thermal indicators and geolocation data to determine likely hotspots. Generating an infrastructure digital twin and using a machine learning classification model, combinations of population cluster pockets for pool testing can be predicted by simulator engine 112. The digital twin can enable infrastructure alternations in response to predictions made by simulator engine 112. As a result, ESM system 100 enables a more rapid, more efficient process for planning resource use and predicting outcomes related to a disease outbreak stemming from a known pathogen.

It is expressly noted that although this disclosure includes a detailed description on cloud computing, implementations of the teachings recited herein are not limited to a cloud computing environment. Rather, embodiments of the present invention are capable of being implemented in conjunction with any other type of computing environment now known or later developed.

Cloud computing is a model of service delivery for enabling convenient, on-demand network access to a shared pool of configurable computing resources (e.g., networks, network bandwidth, servers, processing, memory, storage, applications, virtual machines, and services) that can be rapidly provisioned and released with minimal management effort or interaction with a provider of the service. This cloud model may include at least five characteristics, at least three service models, and at least four deployment models.

Characteristics are as follows:

On-demand self-service: a cloud consumer can unilaterally provision computing capabilities, such as server time and network storage, as needed automatically without requiring human interaction with the service's provider.

Broad network access: capabilities are available over a network and accessed through standard mechanisms that promote use by heterogeneous thin or thick client platforms (e.g., mobile phones, laptops, and PDAs).

Resource pooling: the provider's computing resources are pooled to serve multiple consumers using a multi-tenant model, with different physical and virtual resources dynamically assigned and reassigned according to demand. There is a sense of location independence in that the consumer generally has no control or knowledge over the exact location of the provided resources but may be able to specify location at a higher level of abstraction (e.g., country, state, or datacenter).

Rapid elasticity: capabilities can be rapidly and elastically provisioned, in some cases automatically, to quickly scale out and rapidly released to quickly scale in. To the consumer, the capabilities available for provisioning often appear to be unlimited and can be purchased in any quantity at any time.

Measured service: cloud systems automatically control and optimize resource use by leveraging a metering capability at some level of abstraction appropriate to the type of service (e.g., storage, processing, bandwidth, and active user accounts). Resource usage can be monitored, controlled, and reported providing transparency for both the provider and consumer of the utilized service.

Service Models are as follows:

Software as a Service (SaaS): the capability provided to the consumer is to use the provider's applications running on a cloud infrastructure. The applications are accessible from various client devices through a thin client interface such as a web browser (e.g., web-based e-mail). The consumer does not manage or control the underlying cloud infrastructure including network, servers, operating systems, storage, or even individual application capabilities, with the possible exception of limited user-specific application configuration settings.

Platform as a Service (PaaS): the capability provided to the consumer is to deploy onto the cloud infrastructure consumer-created or acquired applications created using programming languages and tools supported by the provider. The consumer does not manage or control the underlying cloud infrastructure including networks, servers, operating systems, or storage, but has control over the deployed applications and possibly application hosting environment configurations.

Infrastructure as a Service (IaaS): the capability provided to the consumer is to provision processing, storage, networks, and other fundamental computing resources where the consumer is able to deploy and run arbitrary software, which can include operating systems and applications. The consumer does not manage or control the underlying cloud infrastructure but has control over operating systems, storage, deployed applications, and possibly limited control of select networking components (e.g., host firewalls).

Deployment Models are as follows:

Private cloud: the cloud infrastructure is operated solely for an organization. It may be managed by the organization or a third party and may exist on-premises or off-premises.

Community cloud: the cloud infrastructure is shared by several organizations and supports a specific community that has shared concerns (e.g., mission, security requirements, policy, and compliance considerations). It may be managed by the organizations or a third party and may exist on-premises or off-premises.

Public cloud: the cloud infrastructure is made available to the general public or a large industry group and is owned by an organization selling cloud services.

Hybrid cloud: the cloud infrastructure is a composition of two or more clouds (private, community, or public) that remain unique entities but are bound together by standardized or proprietary technology that enables data and application portability (e.g., cloud bursting for load-balancing between clouds).

A cloud computing environment is service oriented with a focus on statelessness, low coupling, modularity, and semantic interoperability. At the heart of cloud computing is an infrastructure comprising a network of interconnected nodes.

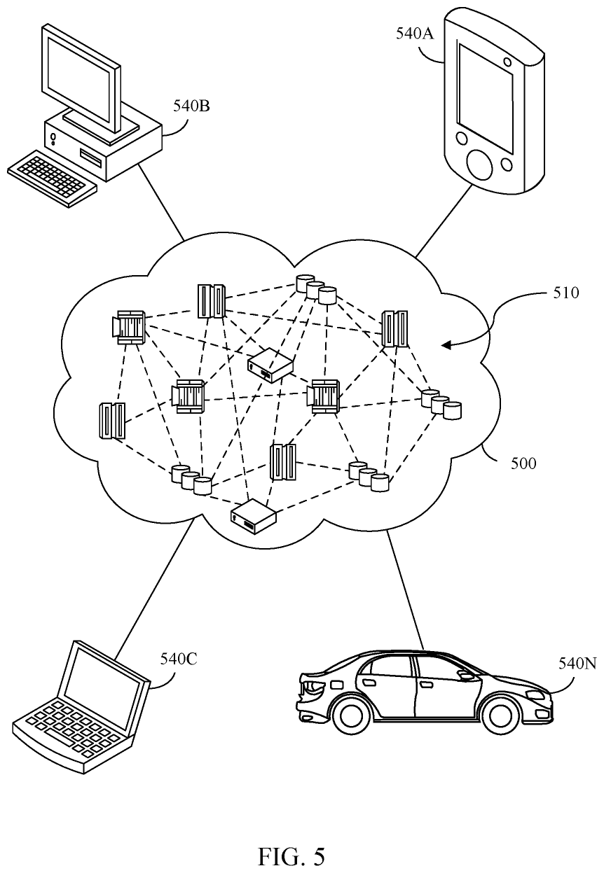

Referring now to FIG. 5, illustrative cloud computing environment 500 is depicted. As shown, cloud computing environment 500 includes one or more cloud computing nodes 510 with which local computing devices used by cloud consumers, such as, for example, personal digital assistant (PDA) or cellular telephone 540a, desktop computer 540b, laptop computer 540c, and/or automobile computer system 540n may communicate. Computing nodes 510 may communicate with one another. They may be grouped (not shown) physically or virtually, in one or more networks, such as Private, Community, Public, or Hybrid clouds as described hereinabove, or a combination thereof. This allows cloud computing environment 500 to offer infrastructure, platforms and/or software as services for which a cloud consumer does not need to maintain resources on a local computing device. It is understood that the types of computing devices 540a-n shown in FIG. 5 are intended to be illustrative only and that computing nodes 510 and cloud computing environment 500 can communicate with any type of computerized device over any type of network and/or network addressable connection (e.g., using a web browser).

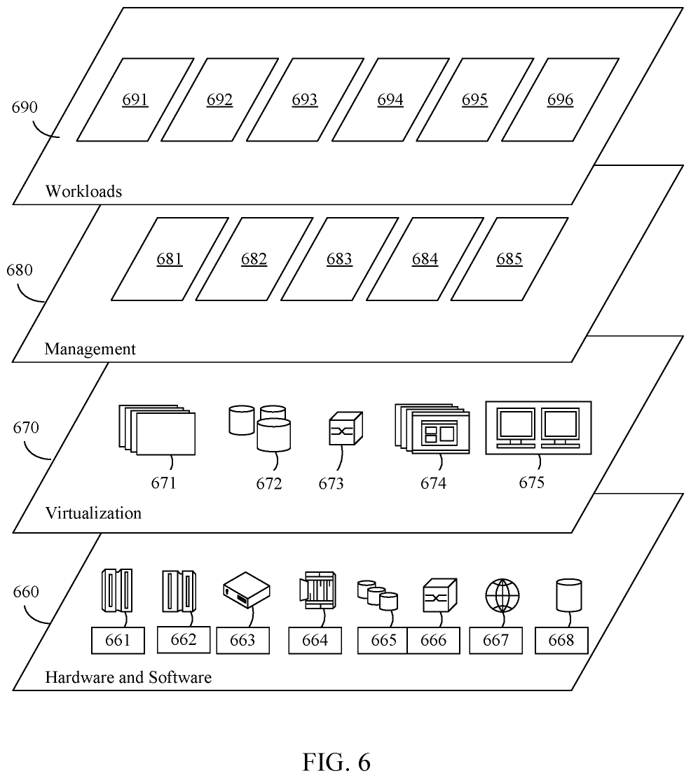

Referring now to FIG. 6, a set of functional abstraction layers provided by cloud computing environment 500 (FIG. 5) is shown. It should be understood in advance that the components, layers, and functions shown in FIG. 6 are intended to be illustrative only and embodiments of the invention are not limited thereto. As depicted, the following layers and corresponding functions are provided:

Hardware and software layer 660 includes hardware and software components. Examples of hardware components include mainframes 661; RISC (Reduced Instruction Set Computer) architecture-based servers 662; servers 663; blade servers 664; storage devices 665; and networks and networking components 666. In some embodiments, software components include network application server software 667 and database software 668.

Virtualization layer 670 provides an abstraction layer from which the following examples of virtual entities may be provided: virtual servers 671; virtual storage 672; virtual networks 673, including virtual private networks; virtual applications and operating systems 674; and virtual clients 675.

In one example, management layer 680 may provide the functions described below. Resource provisioning 681 provides dynamic procurement of computing resources and other resources that are utilized to perform tasks within the cloud computing environment. Metering and Pricing 682 provide cost tracking as resources are utilized within the cloud computing environment, and billing or invoicing for consumption of these resources. In one example, these resources may include application software licenses. Security provides identity verification for cloud consumers and tasks, as well as protection for data and other resources. User portal 683 provides access to the cloud computing environment for consumers and system administrators. Service level management 684 provides cloud computing resource allocation and management such that required service levels are met. Service Level Agreement (SLA) planning and fulfillment 685 provide pre-arrangement for, and procurement of, cloud computing resources for which a future requirement is anticipated in accordance with an SLA.

Workloads layer 690 provides examples of functionality for which the cloud computing environment may be utilized. Examples of workloads and functions which may be provided from this layer include: mapping and navigation 691; software development and lifecycle management 692; virtual classroom education delivery 693; data analytics processing 694; transaction processing 695; and EMS system 696.

FIG. 7 illustrates a schematic of an example of a computing node 700. In one or more embodiments, computing node 700 is an example of a suitable cloud computing node. Computing node 700 is not intended to suggest any limitation as to the scope of use or functionality of embodiments of the invention described herein. Computing node 700 is capable of performing any of the functionality described within this disclosure.

Computing node 700 includes a computer system 712, which is operational with numerous other general-purpose or special-purpose computing system environments or configurations. Examples of well-known computing systems, environments, and/or configurations that may be suitable for use with computer system 712 include, but are not limited to, personal computer systems, server computer systems, thin clients, thick clients, hand-held or laptop devices, multiprocessor systems, microprocessor-based systems, set top boxes, programmable consumer electronics, network PCs, minicomputer systems, mainframe computer systems, and distributed cloud computing environments that include any of the above systems or devices, and the like.

Computer system 712 may be described in the general context of computer system-executable instructions, such as program modules, being executed by a computer system. Generally, program modules may include routines, programs, objects, components, logic, data structures, and so on that perform particular tasks or implement particular abstract data types. Computer system 712 may be practiced in distributed cloud computing environments where tasks are performed by remote processing devices that are linked through a communications network. In a distributed cloud computing environment, program modules may be located in both local and remote computer system storage media including memory storage devices.

As shown in FIG. 7, computer system 712 is shown in the form of a general-purpose computing device. The components of computer system 712 may include, but are not limited to, one or more processors 716, a memory 728, and a bus 718 that couples various system components including memory 728 to processor 716. As defined herein, “processor” means at least one hardware circuit configured to carry out instructions. The hardware circuit may be an integrated circuit. Examples of a processor include, but are not limited to, a central processing unit (CPU), an array processor, a vector processor, a digital signal processor (DSP), a field-programmable gate array (FPGA), a programmable logic array (PLA), an application specific integrated circuit (ASIC), programmable logic circuitry, and a controller.

The carrying out of instructions of a computer program by a processor comprises executing or running the program. As defined herein, “run” and “execute” comprise a series of actions or events performed by the processor in accordance with one or more machine-readable instructions. “Running” and “executing,” as defined herein refer to the active performing of actions or events by the processor. The terms run, running, execute, and executing are used synonymously herein.

Bus 718 represents one or more of any of several types of bus structures, including a memory bus or memory controller, a peripheral bus, an accelerated graphics port, and a processor or local bus using any of a variety of bus architectures. By way of example only, and not limitation, such architectures include Industry Standard Architecture (ISA) bus, Micro Channel Architecture (MCA) bus, Enhanced ISA (EISA) bus, Video Electronics Standards Association (VESA) local bus, Peripheral Component Interconnect (PCI) bus, and PCI Express (PCIe) bus.

Computer system 712 typically includes a variety of computer system-readable media. Such media may be any available media that is accessible by computer system 712, and may include both volatile and non-volatile media, removable and non-removable media.

Memory 728 may include computer system readable media in the form of volatile memory, such as random-access memory (RAM) 730 and/or cache memory 732. Computer system 712 may further include other removable/non-removable, volatile/non-volatile computer system storage media. By way of example, storage system 734 can be provided for reading from and writing to a non-removable, non-volatile magnetic media and/or solid-state drive(s) (not shown and typically called a “hard drive”). Although not shown, a magnetic disk drive for reading from and writing to a removable, non-volatile magnetic disk (e.g., a “floppy disk”), and an optical disk drive for reading from or writing to a removable, non-volatile optical disk such as a CD-ROM, DVD-ROM or other optical media can be provided. In such instances, each can be connected to bus 718 by one or more data media interfaces. As will be further depicted and described below, memory 728 may include at least one program product having a set (e.g., at least one) of program modules that are configured to carry out the functions of embodiments of the invention.

Program/utility 740, having a set (at least one) of program modules 742, may be stored in memory 728 by way of example, and not limitation, as well as an operating system, one or more application programs, other program modules, and program data. Each of the operating system, one or more application programs, other program modules, and program data or some combination thereof, may include an implementation of a networking environment. Program modules 742 generally carry out the functions and/or methodologies of embodiments of the invention as described herein. For example, one or more of the program modules may include EMS system 696 or portions thereof.

Program/utility 740 is executable by processor 716. Program/utility 740 and any data items used, generated, and/or operated upon by computer system 712 are functional data structures that impart functionality when employed by computer system 712. As defined within this disclosure, a “data structure” is a physical implementation of a data model's organization of data within a physical memory. As such, a data structure is formed of specific electrical or magnetic structural elements in a memory. A data structure imposes physical organization on the data stored in the memory as used by an application program executed using a processor.

Computer system 712 may also communicate with one or more external devices 714 such as a keyboard, a pointing device, a display 724, etc.; one or more devices that enable a user to interact with computer system 712; and/or any devices (e.g., network card, modem, etc.) that enable computer system 712 to communicate with one or more other computing devices. Such communication can occur via input/output (I/O) interfaces 722. Still, computer system 712 can communicate with one or more networks such as a local area network (LAN), a general wide area network (WAN), and/or a public network (e.g., the Internet) via network adapter 720. As depicted, network adapter 720 communicates with the other components of computer system 712 via bus 718. It should be understood that although not shown, other hardware and/or software components could be used in conjunction with computer system 712. Examples, include, but are not limited to: microcode, device drivers, redundant processing units, external disk drive arrays, RAID systems, tape drives, and data archival storage systems, etc.

While computing node 700 is used to illustrate an example of a cloud computing node, it should be appreciated that a computer system using an architecture the same as or similar to that described in connection with FIG. 7 may be used in a non-cloud computing implementation to perform the various operations described herein. In this regard, the example embodiments described herein are not intended to be limited to a cloud computing environment. Computing node 700 is an example of a data processing system. As defined herein, “data processing system” means one or more hardware systems configured to process data, each hardware system including at least one processor programmed to initiate operations and memory.

Computing node 700 is an example of computer hardware. Computing node 700 may include fewer components than shown or additional components not illustrated in FIG. 7 depending upon the particular type of device and/or system that is implemented. The particular operating system and/or application(s) included may vary according to device and/or system type as may the types of I/O devices included. Further, one or more of the illustrative components may be incorporated into, or otherwise form a portion of, another component. For example, a processor may include at least some memory.

Computing node 700 is also an example of a server. As defined herein, “server” means a data processing system configured to share services with one or more other data processing systems. As defined herein, “client device” means a data processing system that requests shared services from a server, and with which a user directly interacts. Examples of a client device include, but are not limited to, a workstation, a desktop computer, a computer terminal, a mobile computer, a laptop computer, a netbook computer, a tablet computer, a smart phone, a personal digital assistant, a smart watch, smart glasses, a gaming device, a set-top box, a smart television and the like. In one or more embodiments, the various user devices described herein may be client devices. Network infrastructure, such as routers, firewalls, switches, access points and the like, are not client devices as the term “client device” is defined herein.

The terminology used herein is for the purpose of describing particular embodiments only and is not intended to be limiting. Notwithstanding, several definitions that apply throughout this document now will be presented.

As defined herein, the singular forms “a,” “an,” and “the” include the plural forms as well, unless the context clearly indicates otherwise.

As defined herein, “another” means at least a second or more.

As defined herein, “at least one,” “one or more,” and “and/or,” are open-ended expressions that are both conjunctive and disjunctive in operation unless explicitly stated otherwise. For example, each of the expressions “at least one of A, B and C,” “at least one of A, B, or C,” “one or more of A, B, and C,” “one or more of A, B, or C,” and “A, B, and/or C” means A alone, B alone, C alone, A and B together, A and C together, B and C together, or A, B and C together.

As defined herein, “automatically” means without user intervention.

As defined herein, “individual” and “member of the population” refer to individual human beings, and relatedly, “individuals” and “members of the population” refer to multiple human beings.

As defined herein, “includes,” “including,” “comprises,” and/or “comprising,” specify the presence of stated features, integers, steps, operations, elements, and/or components, but do not preclude the presence or addition of one or more other features, integers, steps, operations, elements, components, and/or groups thereof.

As defined herein, “if” means “in response to” or “responsive to,” depending upon the context. Thus, the phrase “if it is determined” may be construed to mean “in response to determining” or “responsive to determining” depending on the context. Likewise the phrase “if [a stated condition or event] is detected” may be construed to mean “upon detecting [the stated condition or event]” or “in response to detecting [the stated condition or event]” or “responsive to detecting [the stated condition or event]” depending on the context.

As defined herein, “one embodiment,” “an embodiment,” “in one or more embodiments,” “in particular embodiments,” or similar language mean that a particular feature, structure, or characteristic described in connection with the embodiment is included in at least one embodiment described within this disclosure. Thus, appearances of the aforementioned phrases and/or similar language throughout this disclosure may, but do not necessarily, all refer to the same embodiment.

As defined herein, the phrases “in response to” and “responsive to” mean responding or reacting readily to an action or event. Thus, if a second action is performed “in response to” or “responsive to” a first action, there is a causal relationship between an occurrence of the first action and an occurrence of the second action. The phrases “in response to” and “responsive to” indicate the causal relationship.

As defined herein, “real time” means a level of processing responsiveness that an individual or system senses as sufficiently immediate for a particular process or determination to be made, or that enables the processor to keep up with some external process.

As defined herein, “substantially” means that the recited characteristic, parameter, or value need not be achieved exactly, but that deviations or variations, including for example, tolerances, measurement error, measurement accuracy limitations, and other factors known to those of skill in the art, may occur in amounts that do not preclude the effect the characteristic was intended to provide.

As defined herein, “user” and “individual” each refer to a human being.

The terms first, second, etc. may be used herein to describe various elements. These elements should not be limited by these terms, as these terms are only used to distinguish one element from another unless stated otherwise or the context clearly indicates otherwise.

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be accomplished as one step, executed concurrently, substantially concurrently, in a partially or wholly temporally overlapping manner, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various embodiments of the present invention have been presented for purposes of illustration and are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A computer-implemented process, comprising:
generating, with a networked computer, a virtual representation of a predetermined geospatial region by communicating in real time via a data communications network with a plurality of sensor-endowed computing nodes that capture and convey sensor-generated data in real time to the networked computer;
deriving, with the networked computer, a causal network mapping biodata onto inferences regarding pathogenic vector dynamics of a known pathogen based on stochastic vector probabilities, wherein the biodata is culled from historical data associated with the predetermined geospatial region;
generating, with the networked computer, a visualization of selected characteristics of the predetermined geospatial region and creating a corresponding epidemiological model based on the historical data;
correlating, with the networked computer, the sensor-generated data and historical data; and
predicting an expected effect of the known pathogen on a population of the geospatial region using a model simulation based on the correlating the sensor-generated data and historical data.

2. The computer-implemented process of claim 1, wherein the virtual representation is a digital twin.

3. The computer-implemented process of claim 2, wherein the digital twin comprises a plurality of interacting sub-twins.

4. The computer-implemented process of claim 1, wherein the model simulation simulates outcomes of a predefined non-pharmaceutical intervention strategy for determining an effectiveness threshold of the non-pharmaceutical intervention strategy.

5. The computer-implemented process of claim 1, further comprising generating an AI model using machine learning, wherein the machine learning is performed using simulated outcomes generated by the simulation model to train and validate the AI model.

6. The computer-implemented process of claim 5, wherein the AI model predicts risk factors associated with pre-existing conditions of members of a population.

7. The computer-implemented process of claim 1, further comprising forecasting healthcare system factors based on the sensor-generated data correlated with the historical data.

8. A system, comprising:
a processor configured to initiate operations including:
generating a virtual representation of a predetermined geospatial region by communicating in real time via a data communications network with a plurality of sensor-endowed computing nodes that capture and convey sensor-generated data in real time to the networked computer;
deriving a causal network mapping biodata onto inferences regarding pathogenic vector dynamics of a known pathogen based on stochastic vector probabilities, wherein the biodata is culled from historical data associated with the predetermined geospatial region;
generating a visualization of selected characteristics of the predetermined geospatial region and creating a corresponding epidemiological model based on the historical data;
correlating the sensor-generated data and historical data; and
predicting an expected effect of the known pathogen on a population of the geospatial region using a model simulation based on the correlating the sensor-generated data and historical data.

9. The system of claim 8, wherein the virtual representation is a digital twin.

10. The system of claim 9, wherein the digital twin comprises a plurality of interacting sub-twins.

11. The system of claim 8, wherein the model simulation simulates outcomes of a predefined non-pharmaceutical intervention strategy for determining an effectiveness threshold of the non-pharmaceutical intervention strategy.

12. The system of claim 8, wherein the processor is configured to initiate further operations including generating an AI model using machine learning, wherein the machine learning is performed using simulated outcomes generated by the simulation model to train and validate the AI model.

13. The system of claim 12, wherein the AI model predicts risk factors associated with pre-existing conditions of members of a population.

14. A computer program product, the computer program product comprising:
one or more computer-readable storage media and program instructions collectively stored on the one or more computer-readable storage media, the program instructions executable by a processor to cause the processor to initiate operations including:
generating a virtual representation of a predetermined geospatial region by communicating in real time via a data communications network with a plurality of sensor-endowed computing nodes that capture and convey sensor-generated data in real time to the networked computer;
deriving a causal network mapping biodata onto inferences regarding pathogenic vector dynamics of a known pathogen based on stochastic vector probabilities, wherein the biodata is culled from historical data associated with the predetermined geospatial region;
generating a visualization of selected characteristics of the predetermined geospatial region and creating a corresponding epidemiological model based on the historical data;
correlating the sensor-generated data and historical data; and
predicting an expected effect of the known pathogen on a population of the geospatial region using a model simulation based on the correlating the sensor-generated data and historical data.

15. The computer program product of claim 14, wherein the virtual representation is a digital twin.

16. The computer program product of claim 15, wherein the digital twin comprises a plurality of interacting sub-twins.

17. The computer program product of claim 14, wherein the model simulation simulates outcomes of a predefined non-pharmaceutical intervention strategy for determining an effectiveness threshold of the non-pharmaceutical intervention strategy.

18. The computer program product of claim 14, wherein the program instructions are executable by the processor to cause the processor to initiate operations further including generating an AI model using machine learning, wherein the machine learning is performed using simulated outcomes generated by the simulation model to train and validate the AI model.

19. The computer program product of claim 18, wherein the AI model predicts risk factors associated with pre-existing conditions of members of a population.

20. The computer program product of claim 14, wherein the program instructions are executable by the processor to cause the processor to initiate operations further including forecasting healthcare system factors based on the sensor-generated data correlated with the historical data.
